(19) United States
(12) Patent Application Publication
(10) Pub. No.: US 2024/0207314 A1
(43) Pub. Date: Jun. 27, 2024

(54) METHODS TO IMPROVE T CELL EFFICACY AND SAFETY BY MODULATING MEDIATORS OF PHAGOCYTOSIS

(71) Applicant: The Board of Trustees of the Leland Stanford Junior University, Stanford, CA (US)

(72) Inventors: Johanna Lena Theruvath, Stanford, CA (US); Crystal Mackall, Stanford, CA (US); Elena Sotillo, Stanford, CA (US)

(21) Appl. No.: 18/288,913

(22) PCT Filed: Apr. 29, 2022
(86) PCT No.: PCT/US22/27022

§ 371 Date: Oct. 30, 2023

Related U.S. Application Data
(60) Provisional application No. 63/182,189, filed on Apr. 30, 2021.

(51) Int. Cl. A61K 35/17; A61K 39/00; A61P 35/00; A61P 37/04; C07K 14/705; C07K 16/28; C07K 16/32

(52) U.S. Cl. CPC A61K 35/17; A61K 39/4611; A61K 39/4613; A61K 39/4631; A61K 39/464406; A61K 39/464412; A61P 35/00; A61P 37/04; C07K 14/70503; C07K 16/2803; C07K 16/32; A61K 2239/13; A61K 2239/21; A61K 2239/22

(57) ABSTRACT

Provided herein are engineered lymphocytes which overexpress one or more anti-phagocytic signaling proteins, and methods of using same to induce an immune response against cancer cells by inhibiting immune clearance of the engineered T cells. Also provided is a method of depleting engineered T cells in a subject by administering to the subject an agent that inhibits the activity of one or more anti-phagocytic signaling proteins expressed by the engineered T cells.

### CROSS-REFERENCE TO RELATED APPLICATIONS

This application claims the benefit of U.S. Provisional Application No. 63/182,189, filed September Apr. 30, 2021, the content of which is herein incorporated by reference in its entirety.

### BACKGROUND OF THE INVENTION

The presence of tumor associated macrophages (TAMs) is generally associated with poor prognosis in solid tumors; thus, modulation and reeducation of the TAM pool has been explored as an antitumor strategy. TAMs are a main contributor to the immunosuppressive tumor microenvironment (TME). The immunosuppressive TME is known to impact T cell function (Xia et al., Front Immunol, 10: 1719, doi:10.3389/fimmu.2019.01719 (2019)). It is well characterized that macrophages interact with T cells through an MHC-TCR dependent manner. Foreign antigen presentation on MHC on antigen-presenting cells like macrophages leads to TCR-dependent T cell activation.

Chimeric antigen receptor (CAR) T cells have revolutionized the treatment of hematologic malignancies but have not yet demonstrated substantial activity in solid tumors (Majzner, R. G. & Mackall, C. L., Nat Med, 25: 1341-1355, doi:10.1038/s41591-019-0564-6 (2019)). Antigen exposure via CAR T cells leads to an MHC-TCR independent T cell activation, and it is much less known what role macrophages play in a TCR independent CAR T cell context.

There remains a need for compositions and methods that modulate the activity and efficacy of therapies that utilize engineered T cells, such as CAR T cells.

### BRIEF SUMMARY OF THE INVENTION

The disclosure provides an engineered lymphocyte that overexpresses one or more anti-phagocytic signaling proteins, as well as a composition comprising such engineered lymphocytes. Also provided is a method of inducing an immune response against one or more cancer cells, which comprises contacting one or more cancer cells with the composition, whereupon an immune response against the one or more cancer cells is induced.

The disclosure also provides a method of inhibiting immune clearance of genetically engineered T cells in a subject, which method comprises: (a) genetically engineering T cells to overexpress one or more anti-phagocytic signaling proteins, and (b) administering the genetically engineered T cells to a subject in need thereof, whereby the one or more anti-phagocytic signaling proteins are overexpressed by the genetically engineered T cells and immune clearance of the genetically engineered T cells is inhibited.

Also provided is a method of depleting engineered T cells in a subject in need of T cell depletion, which method comprises administering to a subject who has received engineered T cells an agent that inhibits the activity of one or more anti-phagocytic signaling proteins expressed by the engineered T cells.

The disclosure further provides the use of the aforementioned engineered lymphocyte or composition for the treatment of cancer, as well as the use of an agent that inhibits the activity of one or more anti-phagocytic signaling proteins for depleting engineered T cells in a subject.

### BRIEF DESCRIPTION OF THE DRAWING(S)

FIG. 1 shows that CD47 blockade depletes CAR T cells and abrogates CAR T cell efficacy. FIG. 1A is schematic illustrating the experimental setup for evaluation of B7-H3 CAR T cells+/−anti-CD47 in a xenograft model of medulloblastoma. FIGS. 1B and 1C are graphs showing bioluminescence signal (FIG. 1B) and Kaplan Meier curve (FIG. 1C) of mice treated as shown in FIG. 1A. FIG. 1D is a schematic illustrating the experimental setup for evaluation of B7-H3 and GD2 CAR T cells+/−anti-CD47 in a xenograft model of osteosarcoma. FIGS. 1E and 1F are graphs showing tumor area (mm2) (FIG. 1E) and Kaplan Meier curve (FIG. 1F) for mice treated as shown in FIG. 1D. FIGS. 1G, 1H, and 1I are graphs showing quantification of human CD45+ cells in the tumor (FIG. 1G), spleen (FIG. 1H), or T-cells in blood (FIG. 1I) of the mice treated as shown in FIG. 1D.

FIG. 2 shows that CD47 blockade depletes CAR T cells by macrophages and thereby abrogates CAR T cell efficacy. FIG. 2A is a graph showing bioluminescence signal from leukemic mice obtained serially. FIG. 2B includes representative IVIS images 17 days after tumor engraftment (13 days after CAR T cell treatment). FIG. 2C is a graph showing bioluminescence signal of CD19 CAR T cells+/−anti-CD47 antibody obtained serially. FIG. 2D is a graph showing % CD3+Tcells in spleen of mice treated with CD19 CAR T cells+/−anti-CD47 antibody. FIG. 2E is a graph showing bioluminescence signal 2 days after CAR T cell treatment+/−anti-CD47 antibody and from mice depleted with macrophages. FIG. 2F shows representative IVIS images.

FIG. 3A is a graph showing weight of mice in grams treated with CD19-CAR T cells (blue), 2.5F CAR T cells (red) or 2.5F CAR T cells+anti-CD47 antibody (green). FIG. 3B is a graph depicting the bioluminescence signal of CD19 CAR T cells and 2.5F CAR T cells+/−anti-CD47 obtained serially. FIG. 3C is a graph showing survival of mice treated as in FIG. 3A. FIG. 3D is a graph showing interferon γ and TNF-α concentration in serum obtained from mice treated as in FIG. 3A on day 4 after CAR T cell treatment.

FIGS. 4A and 4B are graphs showing cell surface expression of CD47 (FIG. 4A) and Calreticulin (FIG. 4B) on different CAR T cells assessed by Flow cytometry after 10 days of in vitro culture.

FIG. 5 is a graph illustrating that CD47 blockade increases CAR T cell phagocytosis by human macrophages. Results from an in vitro phagocytosis assay of different CAR T cells and human macrophages+/−anti-CD47 are shown. % phagocytozing macrophages were assessed by Flow cytometry (phagocytozing macrophages/all macrophages).

FIG. 6A is a schematic of “Eat me” and “Don't eat me” signals interacting with receptors on macrophages. FIGS. 6B and 6C are graphs showing the fold change expression of Eat me (FIG. 6B) and Don't eat me (FIG. 6C) signals on CAR T cells after activation compared to unstimulated control. FIG. 6D is a graph showing that CD40LG is upregulated upon activation.

FIG. 7A is a schematic of the manufacture of CD47-overexpressing CAR T cells by multiple rounds of retroviral transfection. FIG. 7B includes graphs showing expression of CD24 or CD47 after retroviral overexpression in CAR T cells targeting CD19, Her2 and B7-H3 as assessed by Flow cytometry. FIG. 7C includes graphs showing CAR expression of CAR T cells+/−CD24/CD47 overexpression.

FIG. 8 shows that CD47-overexpressing CAR T cells demonstrate superior efficacy in vivo. FIG. 8A is a graph showing the bioluminescence signal of mice treated with 3×10⁶ CD19 CAR T cells, CD19-47 CAR T cells, Mock T cells or Mock-47 T cells. FIG. 8B includes corresponding IVIS images of tumors in FIG. 8A. FIG. 8C includes graphs showing quantification of CD4+ and CD8+ T cells in blood of mice 43 days after CAR T administration. FIG. 8D is a graph showing the bioluminescence signal of mice treated with 0.1×10⁶ CD19 CAR T cells, CD19-47 CAR T cells or 3×10⁶ Mock T cells or Mock-47 T cells. FIG. 8E includes corresponding IVIS images of tumors in FIG. 8D. FIG. 8F includes graphs showing quantification of CD4+ and CD8+ T cells in blood of mice 43 days after CAR T administration.

FIG. 9 illustrates that CD47 overexpressing CAR T cells demonstrate superior efficacy in vivo against a solid tumor model of osteosarcoma. FIG. 9A is a schematic of the in vivo set up of the osteosarcoma xenograft model. FIGS. 9B, 9C, and 9D are graphs showing tumor area (mm²) of osteosarcoma bearing mice treated with either Mock-, Her2- or Her2-CD47 CAR T cells (FIG. 9B) CD4+ T cells (FIG. 9C), or CD8+ T cells (FIG. 9D) in the blood of the mice 10 days after CAR T cell treatment.

FIG. 10 shows that CD47 overexpressing CAR T cells perform similar to regular CAR T cells in absence of macrophages. FIGS. 10A-10C are graphs which show IL-2 secretion of T cells+/−CD47 in response to antigen-positive tumor cells. FIGS. 10D-10F are graphs which show INFgamma secretion of CAR T cells and CD47-overexpressing CAR T cells in response to antigen-positive tumor cells. FIGS. 10G and 10H are graphs showing the killing capacity of CD19-BBz and CD47-overexpressing CD19-BBz CAR T cells. FIGS. 10I and 10J are graphs showing the killing capacity of CD19-28z and CD47-overexpressing CD19-28z CAR T cells.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein, the term “antigen binding domain” refers to a molecular moiety (e.g., part of a CAR) that recognizes and binds to an antigen (e.g., antibody, antibody fragment, aptamer, receptor, cytokine, surface protein, or another antibody-based or non-antibody-based binding element). In particular embodiments, antigens can be of any nature including, but not limited to, proteins, carbohydrates, lipids, and/or synthetic molecules.

As used herein, the term “activation domain” refers to a molecular moiety (e.g., part of a CAR) that interacts with immune cells (e.g., T cell receptor (TCR)) and induces a positive or negative immunomodulatory signal. Illustrative examples of positive immunomodulatory signals include signals that induce cell proliferation, cytokine secretion, or cytolytic activity. Illustrative examples of negative immunomodulatory signals include signals that inhibit cell proliferation, inhibit the secretion of immunosuppressive factors, or induce cell death.

As used herein, the term “native immune cell” refers to an immune cell that naturally occurs in the immune system of a subject. Illustrative examples include, but are not limited to, T cells, natural killer (NK) cells, NKT cells, B cells, and dendritic cells.

As used herein, the terms “engineered immune cell” and “genetically engineered immune cell” refers to an immune cell (e.g., lymphocyte, T cell, NK cell, NKT cell, B cell, dendritic cell, etc.) that is genetically engineered.

As used herein, the term “co-stimulatory domain” or “co-stimulatory signaling domain” refers to a signaling domain of a co-stimulatory molecule. In particular aspects, it refers to a domain that provides additional signals to the immune cell in conjunction with an activation domain. Co-stimulatory molecules are cell surface molecules other than antigen receptors or Fc receptors that provide a second signal required for efficient activation and function of T lymphocytes upon binding to antigen. Illustrative examples of such co-stimulatory molecules include CD27, CD28, 4-1BB (CD137), OX40 (CD134), CD30, CD40, ICOS (CD278), LFA-1, CD2, CD7, LIGHT, NKD2C, CD70, CD80, CD86, and CD83.

The term “chimeric antigen receptor” (“CAR”) refers to a recombinant polypeptide construct comprising at least an extracellular antigen binding domain, a transmembrane domain and an intracellular signaling domain. Upon binding to their target (e.g., displayed on a cancer cell), CARs typically modify the immune response of the immune cells on which they are displayed.

The term “intracellular signaling domain,” when used in reference to a cell surface receptor or a CAR, is a moiety responsible for activation or inhibition of at least one function of the cell upon which the receptor or CAR is displayed. The term “effector function” refers to a specialized function of a cell. For example, effector function of a T cell includes cytolytic activity or helper activity including the secretion of cytokines. Thus, the term “intracellular signaling domain” refers to the portion of a protein which transduces the effector function signal and directs the cell to perform a specialized function. To the extent that a truncated portion or variant of a native intracellular signaling domain is active, such a polypeptide may be used in place of the full native chain, as long as it transduces the effector function signal. The term intracellular signaling domain includes any truncated or variant portion of a polypeptide sequence sufficient to transduce the effector function signal. Examples of intracellular signaling domains include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Cytoplasmic signaling sequences that act in a stimulatory manner comprise signaling motifs which are known as immunoreceptor tyrosine-based activation motifs (ITAMs). Examples of ITAM-containing cytoplasmic signaling sequences include those derived from TCR zeta, FcR gamma, FcR beta, CD3 gamma, CD3 delta, CD3 epsilon, CD3 zeta, CD5, CD22, CD79a, CD79b, and CD66d.

As used herein, the term “transmembrane domain,” when used in reference to a cell surface receptor or a CAR, is a moiety that spans the plasma membrane of the cell and is connected to both the intracellular signaling domain and the extracellular antigen binding domain. A transmembrane domain may be derived either from a natural or from a synthetic source. Where the source is natural, the domain may be derived from any membrane-bound or transmembrane protein, for example, the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, etc. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular signaling domain. A glycine-serine doublet provides a particularly suitable linker.

As used herein, an “immune response” refers to the action of one or more cells of the immune system (e.g., T lymphocytes, B lymphocytes, natural killer (NK) cells, macrophages, eosinophils, mast cells, dendritic cells, neutrophils, etc.) and soluble macromolecules produced by any of these cells or the liver (e.g., antibodies, cytokines, and complement) that results in selective targeting, binding to, damage to, destruction of, and/or elimination from a subject of invading pathogens, cells or tissues infected with pathogens, or cancerous cells or other abnormal/diseased-associated cells.

As used herein, the term “immunotherapy” refers to the treatment or prevention of a disease or condition by a method comprising inducing, enhancing, suppressing or otherwise modifying an immune response.

The terms “overexpress” and “overexpression,” as used herein, refer to the expression of a gene beyond normal (or wild-type) levels, or to expression of a gene in a cell type or developmental stage or condition in which it normally is not expressed. Overexpression is also referred to in the art as “misexpression” and “ectopic expression.” A gene is overexpressed if the expression is increased by at least about 20% (e.g., 25%, 30%, 35%, 40%, 45%, 50%, 55%, 60%, 65%, 70%, 75%, 80%, 85%, 90°/%, 95%, 99%, 100%, 200%, 300%, 500%, or more) as compared to a reference level, control, or wild-type expression level. Levels of expression can be determined according to any of many acceptable protocols known in the art that measure the abundance of encoding RNA (e.g., mRNA), such as quantitative or semi-quantitative polymerase chain reaction (PCR) or northern blot. In other embodiments applicable to protein-coding genes, the expression can be quantified in terms of amount of target protein detected, such as by western blot.

As used herein, the term “adoptive cell transfer” (“ACT”) is the transfer of cells into a patient. The cells may have originated from the patient or from another individual or cell line. The cells are most commonly derived from the immune system, with the goal of improving immune functionality or eliciting a desired immune response. In some embodiments, cells are extracted from a subject, genetically modified (e.g., to express a desired construct (e.g., CAR)), cultured in vitro, and returned to the subject.

As used herein, the term “antibody” refers to a whole antibody molecule or a fragment thereof (e.g., fragments such as scFv, Fab, Fab′, and F(ab′)₂), unless specified otherwise; an antibody may be a polyclonal or monoclonal antibody, a chimeric antibody, a humanized antibody, a human antibody, etc. In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol., 196:901-917; or Chothia, C. et al. Nature, 342:878-883 (1989). The term “CDR” refers to a CDR from either the light or heavy chain, unless otherwise specified.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody (see. e.g., Hudson et al., Nat. Med., 9: 129-134 (2003)). In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen binding region. Exemplary single chain antibodies are discussed in detail in WO 88/01649 and U.S. Pat. Nos. 4,946,778 and 5,260,203. In certain instances, a single variable region (e.g., a heavy chain variable region or a light chain variable region) may have the ability to recognize and bind antigen. Other antibody fragments will be understood by skilled artisans.

The term “antigen binding site” refers to a portion of an antibody capable of specifically binding an antigen. In certain embodiments, an antigen binding site is provided by one or more antibody variable regions.

The term “epitope” refers to any polypeptide determinant capable of specifically binding to an immunoglobulin, a T cell or B cell receptor, or any interacting protein, such as a surface protein. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position. In some embodiments, peptides or polypeptides herein comprise a minimum sequence identity to a base sequence.

As used herein, the terms “administration” and “administering” refer to the act of giving a drug, prodrug, therapeutic, or other agent to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

The disclosure is predicated, at least in part, on the discovery that blockade of anti-phagocytic signals (also referred to as “don't eat me signals”) expressed on the surface of T cells (e.g., CAR T cells) leads to macrophage-mediated elimination of T cells. Conversely, overexpression of such antiphagocytic signals on T cells enhance their expansion and persistence, leading to superior activity (e.g., in tumor models).

Phagocytosis is a balancing act determined by pro- and anti-phagocytic signals. Pro-phagocytic or “eat me” signals like calreticulin and phosphatidylserine are physiologically expressed on dying cells to indicate that they should be removed by macrophages. Healthy cells express anti-phagocytic or “don't eat me” signals on the surface like CD47, CD24 and CD31 (PECAM-1) to avoid macrophage-mediated phagocytosis (Li, W., J Cell Physiol, 227: 1291-1297, doi:10.1002/jcp.22815 (2012)). CD47 interacts with signal regulatory protein alpha (SIRPα) and CD24 binds to sialic-acid-binding Ig-like lectin 10 (siglec10) on phagocytes, resulting in the negative regulation of phagocytosis. Cancer cells overexpress CD47 and CD24, which enables immune evasion from macrophages (Jaiswal et al., Cell, 138: 271-285, doi:10.1016/j.cell.2009.05.046 (2009); Barkal et al., Nature, 572: 392-396, doi:10.1038/s41586-019-1456-0 (2019)). Recently, the blocking of CD47 and CD24 has been shown to induce macrophages and microglia to tumor cell phagocytosis in preclinical models and in a Phase 1 clinical trial (Barkal et al., supra; Hutter et al., Proc Natl Acad Sci USA, 116: 997-1006, doi:10.1073/pnas.1721434116 (2019); Mohanty et al., Mol Oncol., 13: 2049-2061, doi:10.1002/1878-0261.12556 (2019); Advani et al., N Engl J Med, 379:1711-1721, doi:10.1056/NEJMoa1807315 (2018); Sikic et al., Clin Oncol, 37: 946-953, doi:10.1200/JCO.18.02018 (2019)), credentialing CD47 and CD24 as potential immunotherapy targets.

In some embodiments, the disclosure provides engineered lymphocytes which overexpress one or more anti-phagocytic signaling proteins. It will be appreciated that “lymphocytes” are a class of white blood cells that bear variable cell-surface receptors for antigens. Lymphocytes are produced in bone marrow and are found in the blood and in lymph tissue. Lymphocytes include natural killer cells (which function in cell-mediated, cytotoxic innate immunity), T cells (for cell-mediated, cytotoxic adaptive immunity), and B cells (for humoral, antibody-driven adaptive immunity). As defined above, a lymphocyte is “engineered” or “genetically engineered” by genetically modifying at least a portion of the genetic material (e.g., genome) present in the lymphocyte.

In some embodiments, the engineered lymphocyte is a T cell, such as a cultured T cell or a T cell obtained directly from a mammal (e.g., a human). If obtained from a mammal, the T cell can be obtained from numerous sources, including but not limited to blood, bone marrow, lymph node, the thymus, or other tissues or fluids. T cells can also be enriched for or purified. The T cell desirably is a human T cell. The T cell can be of any developmental stage, including but not limited to, a CD4+/CD8+ double positive T cell, a CD4+ helper T cell, e.g., Th, and Th2 cells, a CD8+T− cell (e.g., a cytotoxic T cell), a tumor infiltrating cell, a memory T cell, a naive T cell, and the like. In one embodiment, the T cell is a CD8+ T cell or a CD4+ T cell. T cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof. T cells which express chimeric antigen receptors (CARs; discussed further herein) are referred to in the art as “CAR T cells,” “CAR-T cells,” or “CART cells.”

In other embodiments, the engineered lymphocyte is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T cells, the NK cell can a cultured NK cell or an NK cell obtained directly from a mammal. If obtained from a mammal, the NK cell can be obtained from numerous sources, including but not limited to blood, bone marrow, lymph node, the thymus, or other tissues or fluids. NK cells can also be enriched for or purified. The NK cell desirably is a human NK cell. NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

Methods for engineering (e.g., genetically engineering) lymphocytes (e.g., T cells) are known in the art. For example, vectors or nucleic acid molecules encoding one or more non-native proteins (e.g., anti-phagocytic signaling proteins, chimeric antigen receptor (CAR) polypeptides, or T cell receptors (TCRs)) may be introduced into a cell (e.g., a lymphocyte) by “transfection,” “transformation,” or “transduction.” “transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and magnetic nanoparticle-based gene delivery (Dobson, J., Gene Ther, 13 (4): 283-7 (2006)). Phage or viral vectors can be introduced into host cells, after growth of infectious particles in suitable packaging cells, many of which are commercially available.

In some embodiments, nucleic acids encoding CAR polypeptides may be inserted into the genetic material of a host cell (e.g., a lymphocyte) using a CRISPR/Cas9 system. CRISPRs are DNA loci comprising short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. The CRISPR/Cas system may be used for gene editing. By delivering the Cas9 protein and appropriate guide RNAs into a cell, the organism's genome can be cut at any desired location. Methods for using CRISPR/Cas9 systems, and other systems, for insertion of a gene into a host cell to produce an engineered cell are described in, for example, U.S. Patent Application Publication 2018/0049412.

The engineered lymphocytes overexpress one or more anti-phagocytic signaling proteins. Phagocytosis is a cellular process for ingesting and eliminating particles larger than about 0.5 μm in diameter, including microorganisms, foreign substances, and apoptotic cells. The prompt and efficient clearance of apoptotic cells by phagocytosis is essential to maintain tissue homeostasis and prevent secondary necrosis. Otherwise, the release of intracellular contents from necrotic cells might trigger inflammation and autoimmune diseases (Nagata et al., Cell, 140(5):619-630 (2010)). In addition, apoptotic cells and phagocytes release tolerogenic signals to reduce immune responses against apoptotic cell-derived self-antigens. Phagocytosis can also be an integral part of physiological processes. To this end, cells undergoing apoptosis must display engulfment signals, such as “find-me” and “eat-me” signals. Engulfment signals are recognized by multiple types of phagocytic machinery in phagocytes, leading to prompt clearance of apoptotic cells. A specialized group of cells including, for example, macrophages, neutrophils, monocytes, dendritic cells, and osteoclasts accomplish phagocytosis with high efficiency.

Pro-phagocytic or “eat me” signals are physiologically expressed on dying cells to indicate that they should be removed by macrophages. Eat-me signals or phagocytosis signaling proteins can be classified into two major categories, membrane-anchored eat-me signals and soluble bridging molecules. Examples of membrane-anchored eat-me signals include, for example, phosphatidylserine and calreticulin. Soluble bridging molecules, such as Gas6 and protein S, are molecular adaptors with at least two binding domains, the receptor-binding domain and phagocytosis prey-binding domain (PPBD) (Caberoy et al., EMBO J.; 29(23): 3898-3910 (2010c)). Soluble bridging molecules, when bound to and displayed on apoptotic cells but not healthy cells through PPBD, serve as eat-me signals to facilitate discriminative clearance of apoptotic cells (Li, supra).

Healthy cells express anti-phagocytic or “don't eat me” signals on the surface to avoid macrophage-mediated phagocytosis (Li, supra). Examples of “don't eat me” signals include, but are not limited to, CD47, CD24, and CD31 (PECAM-1). As discussed above, cancer cells can evade phagocytosis via overexpression of CD47 and CD24. Thus, in some embodiments, the engineered lymphocytes described herein may overexpress one or more of CD47, CD24 and CD31. For example, the engineered lymphocyte overexpresses CD47.

CD47 is an immunoglobulin that is overexpressed on the surface of many types of cancer cells. CD47 forms a signaling complex with signal-regulatory protein α (SIRPα), enabling the escape of these cancer cells from macrophage-mediated phagocytosis. In recent years, CD47 has been shown to be highly expressed by various types of solid tumors and to be associated with poor patient prognosis in various types of cancer. A growing number of studies have demonstrated that inhibiting the CD47-SIRPα signaling pathway promotes the adaptive immune response and enhances the phagocytosis of tumor cells by macrophages. Indeed, blocking CD47-SIRPα interaction with an anti-CD47 antibody or soluble SIRPα variants can promote tumor cell phagocytosis, and at the same time, trigger antitumor T cell immune response (see, e.g., Weiskopf et al., Science; 341: 88-91 (2013); Tseng et al., Proc Natl Acad Sci USA; 110: 11103-11108 (2013); and Liu et al., Nat Med; 21: 1209-1215 (2015)). As detailed below, the inventors of the disclosed subject matter have demonstrated for the first time that blocking the CD47-SIRPα signaling pathway on engineered T cells (e.g., CAR T cells) also marks them for phagocytosis, leading to their rapid and efficient depletion. Conversely, the inventors have also demonstrated that overexpression of anti-phagocytic signals like CD47 on engineered T cells increases expansion, persistence, and potency of the engineered T cells by diminishing macrophage mediated clearance.

In some embodiments, the engineered lymphocyte expresses a chimeric antigen receptor (CAR) polypeptide. The CAR polypeptide desirably comprises an antigen binding domain, a transmembrane domain, at least one co-stimulatory signaling domain, and an intracellular signaling domain. In some embodiments, the CAR further comprises a hinge domain (e.g., extracellular or intracellular hinge) or other linker between domains.

The antigen binding domain of the CAR described herein typically is the extracellular portion of the CAR. The choice of antigen binding domain depends upon the type and number of ligands that define the surface of a target cell. For example, the antigen binding domain may be chosen to recognize a ligand that acts as a cell surface marker on target cells associated with a particular disease state. Cell surface markers that may act as ligands for the antigen binding domain in the CAR of include antigens or proteins associated with viral, bacterial and parasitic infections, or antigens or proteins expressed by diseased cells (e.g., autoimmune disease and cancer).

In some embodiments, the CAR targets cancer cells by displaying an antigen binding domain that specifically binds to an antigen expressed on a cancer cell. The terms “cancer-specific antigen (CSA)” and “tumor-specific antigen (TSA)” are used interchangeably herein and refer to a protein, carbohydrate, or other molecule that is uniquely expressed by and/or displayed on cancer cells and is not expressed by or displayed on other cells in the body (e.g., normal healthy cells). In contrast, the terms “cancer-associated-antigen (CAA)” and “tumor-associated-antigen (TAA)” are used interchangeably herein and refer to a protein, carbohydrate, or other molecule that is not uniquely expressed by or displayed on a tumor cell and instead is also expressed on normal cells under certain conditions.

The selection of the antigen binding domain depends on the particular type of cancer to be treated, and the type of antigens expressed by the cancer cells. Cancer-specific antigens and cancer-associated antigens are well known in the art. In some embodiments, the CSA or CAA comprises one or more antigenic cancer epitopes associated with a malignant cancer or tumor, a metastatic cancer or tumor, or a leukemia. Examples of suitable CSA and CAAs that may be targeted by the CAR polypeptide include, but are not limited to H1ER2, epidermal growth factor receptor (EGFR), vascular endothelial growth factor receptor (VEGFR), interleukin-4 (IL-4), αvβ3 integrin, insulin-like growth factor receptor 1 (IGFR1), insulin-like growth factor receptor 2 (IGFR1), folate receptor, transferrin receptor, estrogen receptor, CXCR4, CD19, interleukin-6 (IL-6), transforming growth factor-beta receptor (TGF-βR), prostate specific membrane antigen (PSMA), α6β1 integrin, IGF1, EphA2, tumor necrosis factor-related apoptosis-inducing ligand (TRAIL), platelet derived growth factor receptor (PDGFR), CD20, HER2/neu, and fibroblast growth factor receptor (FGFR). Other cancer-specific antigens are described in, e.g., Zeromski J., Arch Immunol Ther Exp (Warsz), 50(2): 105-110 (2002); and Boonstra et al., Biomarkers in Cancer, 8: 119-133 (2016); doi:10.4137/BIC.S38542.

In some embodiments, the antigen binding domain of a CAR comprises a variable region of a monoclonal antibody, or antigen binding fragment thereof, that specifically binds to a CSA or CAA. For example, the antigen binding domain may comprise a light chain variable region, a heavy chain variable region, both a light chain variable region and a heavy chain variable region, or antigen-binding fragments thereof, of a monoclonal antibody that specifically binds to a CSA or CAA. A number of monoclonal antibodies that bind to cancer-specific antigens have been approved to treat a variety of different cancers, any of which may be employed in the CAR antigen binding domain. Such monoclonal antibodies include, but are not limited to, trastuzumab (HERCEPTIN®, Genentech, Inc.), cetuximab (ERBITUX®, Eli Lilly and Company), panitumumab (VECTIBIX®, Amgen, Inc.), rituximab (RITUXAN®, Genentech, Inc.), and bevacizumab (AVASTIN®, Genentech, Inc.). The disclosure is not limited to these particular antibodies, however, and any antibody, or antigen-binding fragment thereof, that binds to a cancer-specific antigen may be included in the CAR. In some embodiments, a CAR comprises an extracellular antigen binding domain that is composed of a single chain variable fragment (scFv) scFv. Single chain variable fragments comprise the light chain variable (VL) region and the heavy chain variable (VH) region of a target antigen-specific monoclonal antibody joined by a flexible linker (Bird et al., Science, 242: 423-426 (1988)).

The CAR polypeptide described herein comprises a transmembrane domain. CARs typically are designed to comprise a transmembrane domain that is fused to the extracellular and intracellular domains of the CAR. In some embodiments, a transmembrane domain is a sequence that is naturally associated with one of the other domains in the CAR. In other embodiments, the transmembrane domain is selected or modified by amino acid substitution to avoid interactions with other CAR domains or cell surface components.

In some embodiments, a transmembrane domain is obtained or derived from a natural or a synthetic source. Where the source is natural, the domain may be derived from any membrane-bound or transmembrane protein. Transmembrane regions of particular use include at least the transmembrane region(s) of known transmembrane proteins, including, but not limited to: the alpha, beta or zeta chain of the T cell receptor, CD8α, CD28, CD3 epsilon, CD45, CD4, CD5, CDS, CD9, CD 16, CD22, CD33, CD37, CD64, CD80, CD86, CD 134, CD137, CD 154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic domain of the CAR. A glycine-serine doublet provides a particularly suitable linker. In some embodiments, the disclosed CAR comprises a CD8a transmembrane domain.

The cytoplasmic domain (also referred to as the intracellular signaling domain, activation domain, etc.) of the CAR is responsible for activation of at least one of the normal effector functions of the immune cell in which the CAR has been introduced. As described above, the term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While the entire intracellular signaling domain of a known protein or protein complex may be employed in certain embodiments, in other embodiments it is not necessary to use the entire chain. To the extent that a truncated portion of a known intracellular signaling domain is used, such truncated portion may be used in place of the intact chain as long as it transduces the effector function signal. Examples of intracellular signaling domains for use in the CARs herein include the cytoplasmic sequences of the T cell receptor (TCR) and co-receptors that act in concert to initiate signal transduction following antigen-receptor engagement, as well as any derivative or variant of these sequences and any synthetic sequence that has the same functional capability.

In some embodiments, signals generated through the TCR alone are insufficient for full activation of the T cell. In such cases, a secondary or co-stimulatory signal also is required for full activation. Thus, in some embodiments, T cell activation is mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of 1TAM containing primary cytoplasmic signaling sequences that are of particular use in the invention include those derived from TCR zeta, FcR gamma, FcR beta, CD3 gamma, CD3 delta, CD3 epsilon, CDS, CD22, CD79a, CD79b, and CD66d.

In some embodiments, the cytoplasmic domain of the CAR comprises a primary signaling sequence (e.g., CD3 zeta signaling domain) alone or in combination with any other desired cytoplasmic domain(s) useful in the context of the CAR. In some embodiments, as described above, the cytoplasmic domain of the CAR may comprise a primary signaling sequence and at least one co-stimulatory signaling domain. Any suitable co-stimulatory signaling domain may be used in the CAR Such co-stimulatory domains are present in, for example, CD27, CD28, 4-4BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

The cytoplasmic signaling sequences within the cytoplasmic signaling portion of the CAR may be linked to each other in a random or specified order. Optionally, a short oligopeptide linker (e.g., between 2 and 25 amino acids in length) may form the linkage. A glycine-serine doublet provides a particularly suitable linker.

Between the extracellular domain and the transmembrane domain of the CAR, or between the cytoplasmic domain and the transmembrane domain of the CAR, there may be incorporated a linker domain. A linker domain of a CAR is an oligo- or polypeptide that functions to link the transmembrane domain to, either the extracellular domain or, the cytoplasmic domain in the polypeptide chain. A linker domain may comprise up to 300 amino acids (e.g., 1, 2, 5, 10, 20, 30, 40, 50, 75, 100, 125, 150, 200, 250, 300, or ranges therebetween (e.g., 10 to 100 amino acids, 25 to 50 amino acids, etc.)).

In another embodiment, the CAR comprises a hinge sequence, which is a short amino acid sequence that promote flexibility of the antigen binding domain (see, e.g., Woof et al., Nat. Rev. Immunol., 4(2): 89-99 (2004)). The hinge sequence may be positioned between the antigen binding domain and the transmembrane domain. The hinge sequence can be any suitable sequence derived or obtained from any suitable molecule (e.g., a hinge sequence derived from a human CD8a molecule or a CD28 molecule).

The engineered lymphocytes described herein are not limited to CAR T cells. Indeed, other therapeutic lymphocytes are known in the art and may be further engineered to overexpress one or more anti-phagocytic signaling proteins. Such lymphocytes include, for example, T cell receptor (TCR)-engineered T (TCR-T) cells (see, e.g., Zhao Lijun, Cao Yu J., Frontiers in Immunology, 10: 2250 (2019); doi.org/10.3389/fimmu.2019.02250; Barrett et al., J Immunol., 195: 755-61 (2015). doi: 10.4049/jimmunol.1500751), and T cells employed in adoptive cell therapies (e.g., tumor infiltrating lymphocytes (TIL) and natural killer cells) (see, e.g., Rohaan et al., Virchows Arch.; 474(4): 449-461 (2019)). Adoptive cell therapies for cancer immunotherapy typically involve infusing tumor-specific cytotoxic T cells into cancer patients with the goal of recognizing, targeting, and destroying tumor cells. Adoptive cell transfer methods to treat various types of cancers are known in the art and disclosed in, for example, Gattinoni et al., Nat. Rev. Immunol, 6(5): 383-393 (2006); June, CH, J. Clin. Invest., 117(6): 1466-76 (2007); Rapoport et al., Blood, 117(3): 788-797 (2011); and Barber et al., Gene Therapy, 18: 509-516 (2011).

The present disclosure is not limited to modification of lymphocytes, but also encompasses the modification of phagocytic cells themselves, like macrophages. For example, macrophages may be modified to suppress their activity (e.g., by reducing the ability to recognize engulfment signals on target cells). It will be appreciated that any aspect of the phagocytic process can be disrupted to inhibit phagocytosis of the engineered lymphocytes described herein.

In some embodiments, a lymphocyte is modified to overexpress a wild type “don't eat me” signal. In other embodiments, the “don't eat me” signal is engineered. For example, engineering CD47 or other “don't eat me” proteins may be employed. The modified proteins may be modified to increase expression or localization levels, increase affinity for a binding partner, increase stability, or other desired modifications.

In some embodiments, a lymphocyte is modified with two or more different “don't eat me” signals. For example, a T cell may be engineered to overexpress CD47 and CD24, CD 47, and CD31, CD24 and CD31, CD24, CD47, and CD31, or other such combinations with other “don't eat me” signals.

Engineered lymphocytes, as described herein, can be formulated into a composition, such as a pharmaceutical composition, and administered to an animal, such as a human. For example, the pharmaceutical composition can comprise a population of engineered T cells or NK cells that overexpress the one or more anti-phagocytic signaling proteins and also express a CAR polypeptide. The composition desirably comprises a carrier, such as a pharmaceutically acceptable carrier. The choice of carrier will be determined in part by the particular engineered lymphocytes used, method of administration, and disease to be treated. For example, the pharmaceutical composition may contain preservatives, such as, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition. Methods for preparing administrable (e.g., parenterally administrable) compositions are known to those skilled in the art and are described in more detail in, for example, Remington: The Science and Practice of Pharmacy, Lippincott Williams & Wilkins; 21st ed. (May 1, 2005).

In some embodiments, the composition can employ time-released, delayed release, and sustained release delivery systems such that the delivery of the composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain embodiments.

In some embodiments, the cells are engineering to express the desired protein from an inducible promoter so that timing and level of expression can be managed through administration of an agent that activates or represses the inducible promoter.

The disclosure further provides a method of inducing an immune response against one or more cancer cells. The method comprises contacting one or more cancer cells with the composition comprising engineered lymphocytes that overexpress one or more anti-phagocytic signaling proteins. The engineered lymphocytes overexpressing one or more anti-phagocytic signaling proteins described herein can be contacted with a population of cancer cells ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. In some embodiments, the method involves ex vivo and in vivo components. In this regard, for example, engineered lymphocytes described above can be cultured ex vivo under conditions to express a CAR, and then directly transferred into a mammal (preferably a human) suffering from cancer.

While the invention is not limited to any particular mechanism of action and an understanding of the mechanism of action is not required to practice the invention, it is believed that overexpression of anti-phagocytic molecules on the surface of engineered lymphocytes (e.g., CAR T cells) render them resistant to clearance by tumor-associated macrophages (TAMs), independently of their state of activation, leading to increased function, persistence, and efficacy. As such, the disclosure also provides a method of inhibiting immune clearance of genetically engineered T cells in a subject, which comprises (a) genetically engineering T cells to overexpress one or more anti-phagocytic signaling proteins, and (b) administering the genetically engineered T cells to a subject in need thereof, whereby the one or more anti-phagocytic signaling proteins are expressed by the genetically engineered T cells and immune clearance of the genetically engineered T cells is inhibited. The terms “immune clearance” or “clearance” may be used interchangeably to refer to the removal of an antigen or pathogen from the bloodstream that follows the initiation of an immune response against the antigen or pathogen. Immune clearance typically involves the formation of antigen-antibody complexes, which are ingested by macrophages and other phagocytic cells. Descriptions of T cells, T cell engineering methods, anti-phagocytic signaling proteins, and components thereof, set forth above also are applicable to those same aspects of the aforementioned method of inhibiting immune clearance of genetically engineered T cells. The disclosure also provides the use of an engineered lymphocyte which overexpresses one or more anti-phagocytic signaling proteins, or a composition comprising same, for the treatment of cancer.

Because certain cancer cells express anti-phagocytic or “don't eat me” signals, such as CD47, CD24 and CD31 (PECAM-1), methods for increasing macrophage activity against cancer cells are under investigation. In this regard, to improve the potency of CAR T cells used to treat certain types of cancer, the combination of an anti-CD47 antibody and CAR T cells has been tested by the present inventors in several tumor models (e.g., medulloblastoma, osteosarcoma and leukemia). Surprisingly, it was observed that treatment with an anti-CD47 antibody led to rapid elimination of CAR T cells in vivo, thereby abrogating CAR T cell efficacy. A barrier to widespread use of CAR T-cell therapy is toxicity, primarily cytokine release syndrome (CRS) and neurologic toxicity. As such, in some embodiments, blocking the activity of anti-phagocytic signals like CD47, CD42, and CD31, may be used to deplete engineered T cells as a “safety switch” in situations when engineered T cells cause toxicity. To this end, the disclosure further provides a method of depleting engineered T cells in a subject, which comprises administering to a subject who has received engineered T cells an agent that inhibits the activity of one or more anti-phagocytic signaling proteins expressed by the engineered T cells. Descriptions of engineered T cells, T cell engineering methods, anti-phagocytic signaling proteins, and components thereof, set forth above also are applicable to those same aspects of the aforementioned method of depleting engineered T cells in a subject.

By “deplete” is meant to diminish in number or quantity. Thus, depletion or reduction of engineered T cells within a particular subject may be partially complete (e.g., 10% or more, 25% or more, 50% or more, or 75% or more), substantially complete (e.g., 85% or more, 90% or more, or 95% or more), or fully complete (e.g., 98% or more, or 99% or more). Any suitable agent that inhibits the activity of one or more anti-phagocytic signaling proteins may be administered to a subject who has received engineered T cells. In some embodiments, the agent may be a small molecule. In other embodiments, the agent may be an antibody, such as a monoclonal antibody. For example, the agent may be a monoclonal antibody that specifically binds to CD47, CD42, and CD31. In a particular embodiment, the agent is an anti-CD47 monoclonal antibody, several of which are known in the art. Exemplary anti-CD47 monoclonal antibodies include, but are not limited to, Hu5F9-G4 (Liu et al., PLoS One; 10(9): e0137345 (2015)), AO-176 (Puro et al., Mol Cancer Ther, 19(3) 835-846 (2020); DOI: 10.1158/1535-7163.MCT-19-1079), and commercially available antibodies (e.g., Cat. No. 127519, BioLegend, Inc., San Diego, CA). A Phase I clinical trial testing the anti-CD47 monoclonal antibody Hu5F9-G4 in advanced solid tumors demonstrated that the antibody was safe and well-tolerated (see, e.g., Sikic et al., Journal of Clinical Oncology, 34, no. 15_suppl (May 20, 2016) 3019-3019).

While the invention is not limited to any particular mechanism of action and an understanding of the mechanism of action is not required to practice the invention, depletion of engineered T cells that induce toxicity in a subject will improve the safety of various adoptive T cell therapies. In this respect, CAR T cells can potentially damage normal tissues by specifically targeting a tumor-associated antigen that is also expressed on those tissues. Cytokine release syndrome (CRS), a systemic inflammatory response caused by cytokines released by infused CAR T cells can lead to widespread reversible organ dysfunction. CRS is the most common type of toxicity caused by CAR T cells. CAR T cells also may induce neurologic toxicity. The disclosure also provides the use of an agent that inhibits the activity of one or more anti-phagocytic signaling proteins (e.g., an anti-CD47 monoclonal antibody) for depleting engineered T cells in a subject.

In accordance with the methods described herein, engineered lymphocytes, the agent that inhibits activity of anti-phagocytic signaling proteins, or a composition comprising either of the foregoing, may be administered to a mammal, such as a human, using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration.

In some embodiments, the subject suffers from a cancer, particularly an epithelial cell cancer (e.g., a carcinoma). Examples of such cancers include, but are not limited to, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma and brain metastases).

In other embodiments, engineered lymphocytes, the agent that inhibits activity of anti-phagocytic signaling proteins, or a composition comprising either of the foregoing, may be administered to a subject suffering from a hematological cancer. Examples of hematological cancers that may be treated by the methods disclosed herein include, but are not limited to, leukemias, including acute leukemias (such as acute lymphocytic leukemia, acute myelocytic leukemia, acute myelogenous leukemia and myeloblasts, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom macroglobulinemia, heavy chain disease, myelodysplastic syndrome, hairy cell leukemia, and myelodysplasia.

Engineered lymphocytes, or a composition comprising same, desirably are administered to a subject in an amount that is effective to treat or prevent certain types of cancer. As used herein, the terms “treatment,” “treating,” and the like refer to obtaining a desired pharmacologic and/or physiologic effect. Preferably, the effect is therapeutic, e.g., the effect partially or completely cures a disease and/or adverse symptom attributable to the disease. To this end, the method comprises administering a “therapeutically effective amount” of a composition comprising engineered lymphocytes. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the e to engineered lymphocytes to elicit a desired response in the individual. Likewise, an agent that inhibits activity of anti-phagocytic signaling proteins (e.g., an anti-CD47 antibody) may be administered to a subject in an amount that is effective to induce phagocytosis of engineered T cells expressing the anti-phagocytic signals.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the disclosure. The desired dosage can be delivered by a single bolus administration of the composition, by multiple bolus administrations of the composition, or by continuous infusion administration of the composition.

The disclosure further encompasses combination therapies with other regimens and/or compounds. Combination therapies may be administered simultaneously with, before, or after the methods described herein. Particular combination therapies include, but are not limited to, chemotherapy, radiation, surgery, hormone therapy, or other types of immunotherapy. Many chemotherapeutics are known in the art and can be used in combination with the disclosed methods. In some embodiments, the chemotherapeutic is selected from mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Specific chemotherapeutic agents include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, fludarabine, navelbine, farnesyl-protein transferase inhibitors, transplatinum, 5-fluorouracil, vincristine, and vinblastin, or any analog or variant of the foregoing and also combinations thereof. In some embodiments, chemotherapy is employed before, during and/or after administration of engineered lymphocytes or other compositions described herein.

In some embodiments, the engineered lymphocytes or other compositions described herein are co-administered with radiotherapy using methods known in the art. In some embodiments, radiotherapy is employed before, during, and/or after administration of the engineered lymphocytes or other compositions described herein.

In some embodiments, the engineered lymphocytes or other compositions described herein are co-administered with another immunotherapy. Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect-cell killing. The antibody may also prevent cancer immunoevasion or immunosuppression. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells, NKT cells, and NK cells. In some embodiments, immunotherapy is employed before, during and/or after administration of or other compositions described herein. In some embodiments, engineered lymphocytes are co-administered with an immune checkpoint inhibitor (e.g., anti-PD1, anti-PDL1, anti-CTLA-4, etc.).

In some embodiments, the engineered lymphocytes or other compositions described herein are administered before, during, and/or after surgery. Surgeries include resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that embodiments herein may be used in conjunction with removal of superficial cancers, precancers, or incidental amounts of normal tissue.

In some embodiments, the engineered lymphocytes or other compositions described herein are co-administered with other agents to improve the therapeutic efficacy of treatment.

In some embodiments, engineered lymphocytes or other compositions described herein are provided as part of a kit or system along with one or more additional components, such as instructions, devices for administration, additional therapeutic agents, diagnostic agents, research agents, etc.

The following examples further illustrate the invention but, of course, should not be construed as in any way limiting its scope.

### EXAMPLES

The following materials and methods were employed in the Examples.

### Cells and Culture Conditions

Human cell lines used in the studies described below include MG63.3 (NCI, NIH, Bethesda, MD), NALM6-GL (University of Pennsylvania, Philadelphia, PA), D425 (Stanford University, Stanford, CA), and 293GP and 293T (NCI, NIH, Bethesda, MD). These cell lines were stably transduced with GFP and firefly luciferase. D425 cells were maintained in serum-free media supplemented with B27 (Thermo Fisher Scientific), EGF, FGF (Shenandoah Biotechnology), human recombinant LIF (Millipore), and Heparin (StemCell Technologies).

All other tumor cell lines were cultured in RPMI-1640, supplemented with 10% heat-inactivated FBS (Gibco, Life Technologies), 10 mM HEPES, 100 U/mL penicillin, 100 sg/ml streptomycin and 2 mM L-glutamine (Gibco, Life Technologies).

### Retroviral Vector Production and T Cell Transduction

Retroviral supernatant was produced via transient transfection of the 293GP packaging cell line as previously described. Briefly, 70% confluent cells were co-transfected via Lipofectamine 2000 (Life Technologies) in 150 mm Poly-D-Lysine culture dishes with plasmids encoding CARs and the RD 114 envelope protein. Media was replaced at 24 and 48 hours post transfection. Viral supernatant was harvested 48 and 72 hours post-transfection and centrifuged to remove cell debris and stored at −80° C. until use.

Primary human T cells were isolated from healthy donors using the ROSETTESEP™ Human T cell Enrichment Kit (Stem Cell Technologies) with buffy coats derived from the Stanford Blood Center and processed according to the manufacturer's protocol using LYMPHOPREP™ density gradient medium and SEPMATE™-50 tubes. Isolated T cells were cryopreserved in CRYOSTOR® CS10 cryopreservation medium (Stem Cell Technologies). Cryopreserved T cells were thawed and activated with Human T-Expander CD3/CD28 Dynabeads (Gibco) at a 3:1 beads:cell ratio in AIM-V media supplemented with 5% FBS, 10 mM HEPES, 2 mM 1-glutamine, 100 U/mL penicillin, and 100 μg/mL streptomycin (Gibco) and with 100 IU/mi of recombinant IL-2 (Preprotech). T cells were transduced with retroviral vector on days 2 and 3 post activation and anti-CD3/CD28 beads were removed on day 5. CAR T cells were maintained at 0.3-1×10⁶ cells per mL in T cell medium with IL2. CAR expression was assessed by Flow Cytometry before an assay. CAR T cells were used for in vitro assays or transferred into mice on day 9-12 post activation.

### Flow Cytometry

Data was collected with an LSR Fortessa X-20 (BD Bioscience) and analyzed using the FLOWJO™ software. Cells were harvested, washed twice with FACS buffer (PBS supplemented with 2% FBS and 0.4% 0.5M EDTA) and stained for 30 minutes in the dark on ice. Cells were washed 3 times with FACS buffer after each incubation step. Cells were gated on viable cells, and singlet discrimination (FSC-A/FSC-H) was performed before assessment of antigen expression.

### ELISA

Cytokine release was assayed by co-incubating 0.1×10⁶ CAR+ T cells and 0.1×10⁶ tumor cells in complete RPMI-1640 in triplicates. At 24 hours, culture media were collected and cytokines were measured using IFNy and IL-2 (BioLegend).

### Killing Assay

For INCUCYTE® killing assays, 0.05×10⁶ GPF-positive tumor cells were plated in triplicates in 96-well flat-bottom plates and co-incubated with CAR-positive T-cells or an equivalent number of Mock control T cells at either 1:1 or 1:8 effector to target ratios in 200 ml RPMI-1640. Plates were imaged every 2-3 hours using the INCUCYTE®ZOOM Live-Cell analysis system (Essen Bioscience) and 4 images per well at 10× zoom were collected at each time point. Total integrated GFP intensity per well was assessed as a quantitative measure of viable, GFP-positive tumor cells. Values were normalized to the starting measurement and plotted over time.

### Macrophage Generation and Stimulation

Primary human donor-derived macrophages were generated as described previously (Martinez, F. O., Current Protocols in Immunology, 96(1): 14.28.1-14.28.23 (2012)). Briefly, leukocyte reduction system (LRS) chambers from anonymous donors were obtained from the Stanford Blood Center. Peripheral monocytes were purified through successive density gradients using Ficoll (Sigma Aldrich) and Percoll (GE Healthcare). Monocytes were then differentiated into macrophages by 7-9 days of culture in IMDM+10% AB human serum (Life Technologies).

### In Vitro Phagocytosis Assay

All in vitro phagocytosis assays reported herein were performed by co-culture target cells and donor-derived macrophages at a ratio of 100,000 target cells to 50:000 macrophages for 1-2 hours in a humidified, 5% CO₂ incubator at 37° C. in ultra-low-attachment 96-well U-bottom plates (Corning) in serum-free RMPT (Life Technologies). CAR T cells with endogenous fluorescence were harvested prior to co-culture. CAR T cell lines lacking endogenous fluorescence with CFSE (Invitrogen) were harvested by suspending cells in PBS (5 μM working solution) as per manufacturer instructions for 20 minutes at 37° C. protected from light and washed twice with 20 mL FBS-containing media before co-culture. Macrophages were harvested from plates using TRYPLE™ Express. Anti-CD47 antibody (Clone B6H12, acquired from BioXcell), was added at a concentration of 10 μg/mL. After co-culture, phagocytosis assays were stopped by placing plates on ice, centrifuged at 400 g for 5 minutes at 4° C., and stained with APC-labeled anti-CD1 lb (Clone M1/70, Biolegend) to identify human macrophages. Assays were analyzed by flow cytometry on an LRSFORTESSA™ Analyzer (BD Biosciences). Phagocytosis was measured as the number of CD11 b+, GFP+ macrophages, quantified as a percentage of the total CD11b+ macrophages. Each phagocytosis reaction (independent donor and experimental group) was performed in a minimum of technical triplicate.

### Mice

Immunodeficient NSG mice (NOD.Cg-Prkdcscid 1l2rgtm1Wjl/SzJ) were purchased from The Jackson Laboratory or bred in-house. Mice used for in vivo experiments were between 6 and 12 weeks old and the ratio of male to female mice was matched in experimental and control groups. All animal studies were carried out according to NCI and Stanford University Animal Care and Use Committee-approved protocols.

### MG63.3 Osteosarcoma In Vivo Models—Depletion Experiments

Animal studies were carried out under protocols approved by Stanford University Animal Care and Use Committee. Cell lines were expanded under standard culture conditions (described above) and harvested with 2 mmol/L EDTA (KD Medical) in PBS (Gibco, Thermo Fisher Scientific) or Trypsin (Gibco, Thermo Fisher Scientific). For MG63.3, 1×10⁶ cells were injected periostal to the tibia. In the direct tumor shrinkage experiments, 1×10⁷ B7-H3 CAR+ T cells, 1×10⁷ GD2 CAR+ T cells or an equivalent number of CD19 CAR T cells were injected intravenously into a tail vein 2-3 weeks after tumor inoculation (once the majority of tumors had an area greater than 75 mm²). 400 μg anti-CD47 antibody (clone B6.H12) was administered ip 3×/week starting 15 days (d15) after tumor engraftment. Tumor growth was measured with digital calipers once to twice weekly, and the tumor area was calculated by multiplying the lengths of the major and minor axes. Mice were euthanized when the tumor exceeded a size set by institutional protocol.

MG63.3 Osteosarcoma In Vivo models—Overexpression Experiments

Tumor cells were prepared and injected as described above. Five weeks after tumor engraftment, mice were randomized and treated with either 0.5×10⁶ Her2, Her2-CD47 or Mock (control) CAR T cells and tumor growth was monitored via caliper measurements.

D425 medulloblastoma in vivo models In brief, mice were anesthetized with 3% isoflurane (Minrad International) in an induction chamber. Anesthesia on the stereotactic frame (David Kopf Instruments) was maintained at 2% isoflurane delivered through a nose adaptor. D425 medulloblastoma cells were injected at coordinates 2 mm posterior to lambda on midline and 2 mm deep into 6- to 10-week-old NOD-SCID gamma mice using a blunt-ended needle (75N, 26 s/2″/2, 5 μL; Hamilton Co.). Using a microinjection pump (UMP-3; World Precision Instruments), 2×10⁴ D425-GFP-Luc cells were injected in a volume of 3 μL at 30 nL/s. After leaving the needle in place for 1 minute, it was retracted at 3 mm/min. Tumor formation was followed by bioluminescence imaging on an IVIS spectrum instrument (Caliper Life Science) in the Stanford Small Animal Imaging Facility and quantified with Living Image software (PerkinElmer). Four days after tumor implantation and after confirmation of tumor formation by bioluminescence, mice were randomized and treated with 1×10⁷ B7-H3 CAR+ T cells or an equivalent number of CD19 CAR T cells (matched for total T-cell dose) intravenously by tail vein injection. 300 μg anti-CD47 antibody (clone B6.H12) was administered ip 3×/week starting four days (d4) after tumor engraftment. Isoflurane-anesthetized animals were imaged using the IVIS system (Caliper Life Sciences) 10 minutes after 3 mg d-luciferin (PerkinElmer) was injected intraperitoneally. Living Image (PerkinElmer) software was used to analyze the IVIS data.

NALM6 Leukemia in vivo models For NALM6-GL, 1×10⁶ tumor cells were transferred to NSG mice by tail vein injection. Three to five days later, CD19 CAR+ T cells, or an equivalent total number of mock/untransduced T cells (+exogenous CD47 when indicated) were transferred intravenously. 250 μg anti-CD47 antibody (clone B6.H12) was administered ip 3×/week starting d4 after tumor engraftment. NALM6-GL leukemia burden was evaluated using the Xenogen IVIS Lumina (Caliper Life Sciences). Mice were injected intraperitoneally with 3 mg d-luciferin (PerkinElmer) and then imaged 4 minutes later with an exposure time of 30 seconds. Luminescence images were analyzed using Living Image software (PerkinElmer).

### In Vivo Monitoring of CAR T Cell Trafficking

NanoLuc activity of injected CAR T cells was assessed by injecting 5 μg (approximately equal to 0.25 mg/kg) furimazine (approximately equal to 40× dilution of Nano-Glo substrate) in 100 μL of sterile PBS intraperitoneally; mice were imaged on an IVIS Spectrum within 7 minutes of injection.

### In Vivo Macrophage Depletion

For macrophage depletion, mice were treated with two iv dosages of Clodronate liposomes 4 and 3 days prior to tumor cell injection. Two days before tumor cell injection anti-CSF1R (CD115) treatment was initiated. CD115 was administered 3×/week intraperitoneally.

### Example 1

This example demonstrates that blockade of CD47 depletes CAR T cells.

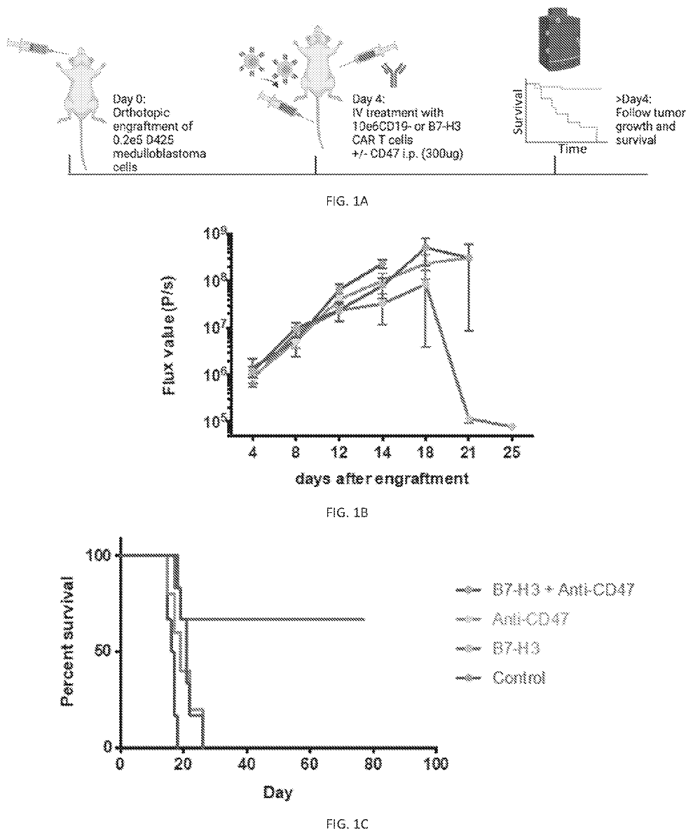

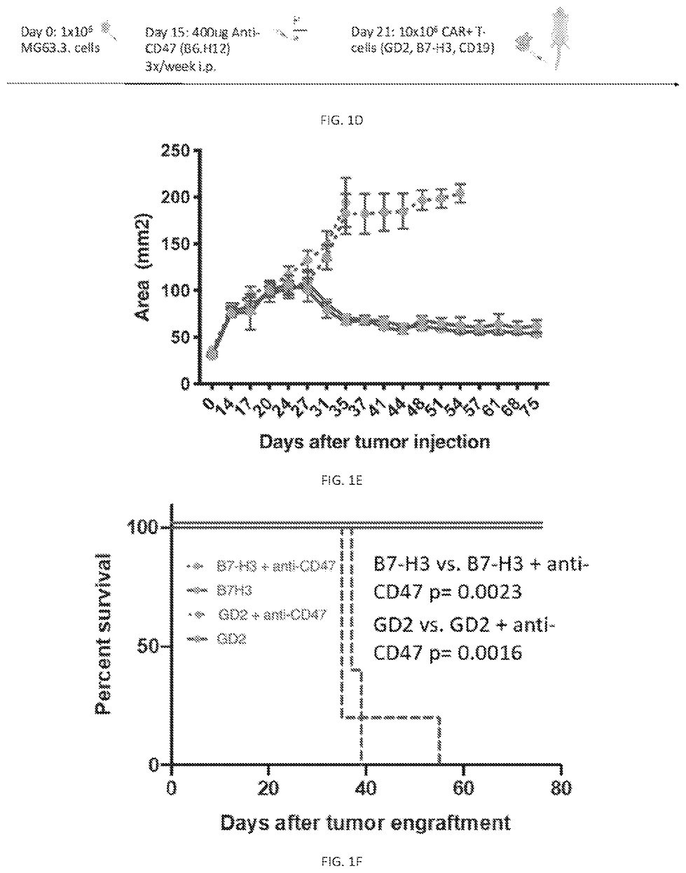

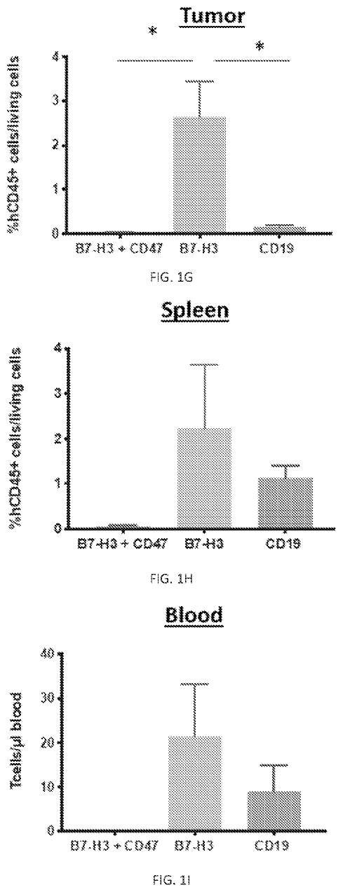

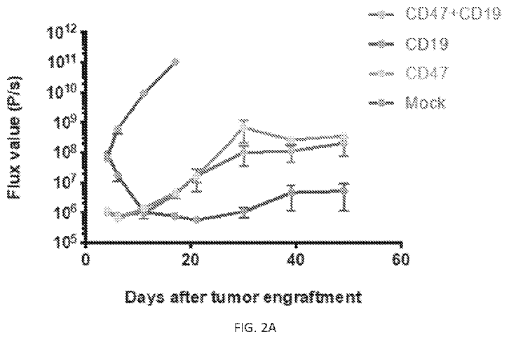

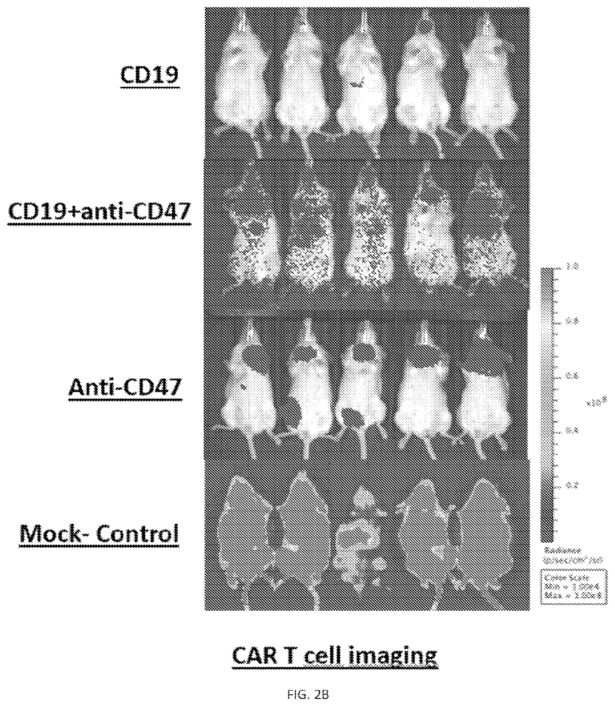

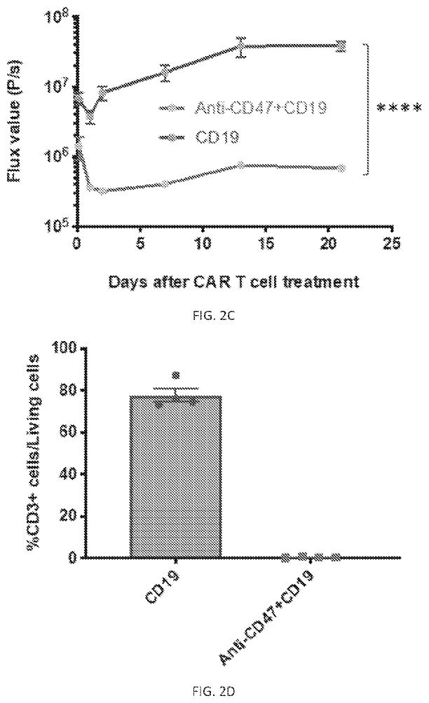

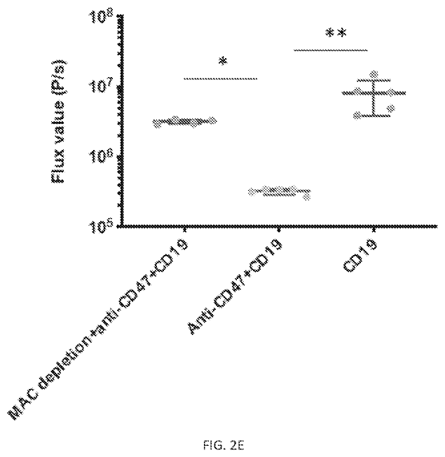

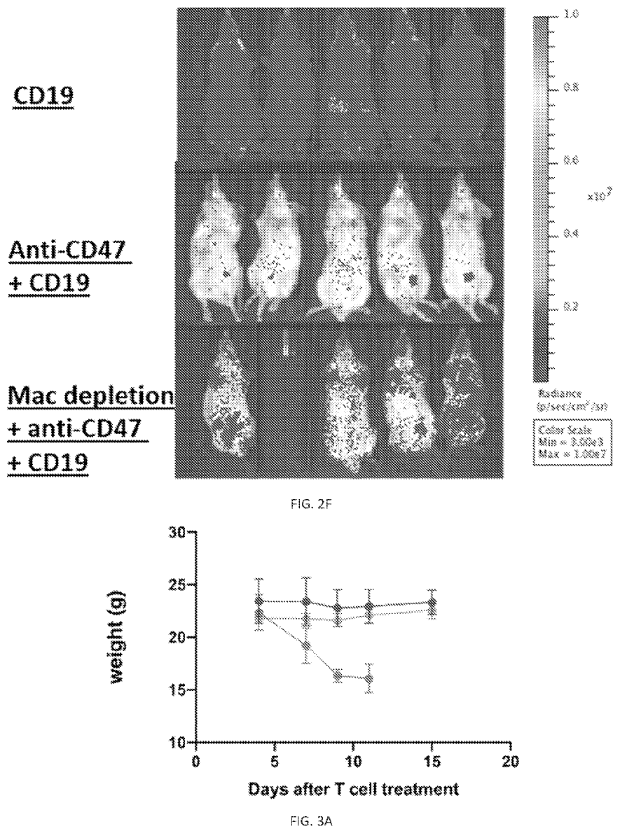

In an effort to increase macrophage activity to improve the potency of CAR T cells, the combination of an anti-CD47 antibody and CAR T cells was tested in several tumor models (e.g., medulloblastoma, osteosarcoma, and leukemia), as shown in FIGS. 1-2. Surprisingly, it was observed that treatment with an anti-CD47 antibody led to rapid elimination of CAR T cells in vivo, thereby abrogating CAR T cell efficacy. CAR T cells were depleted not only from the tumor, but from the spleen and blood of treated mice.

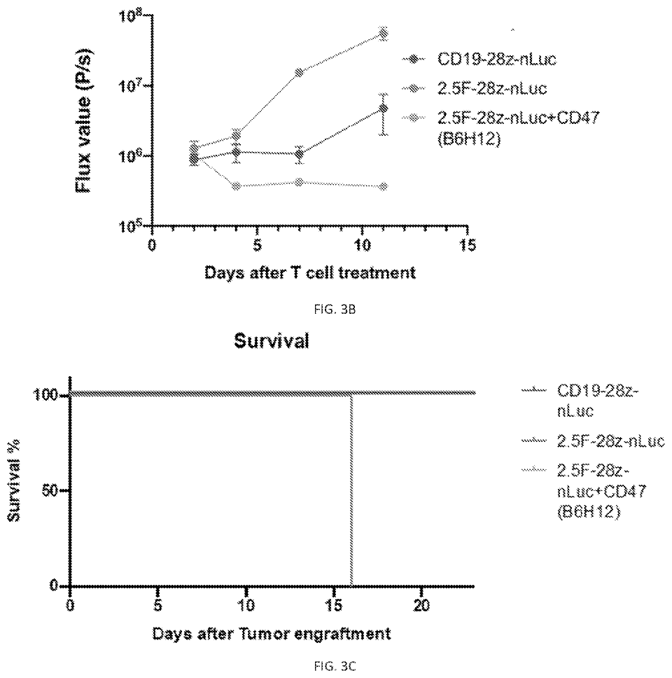

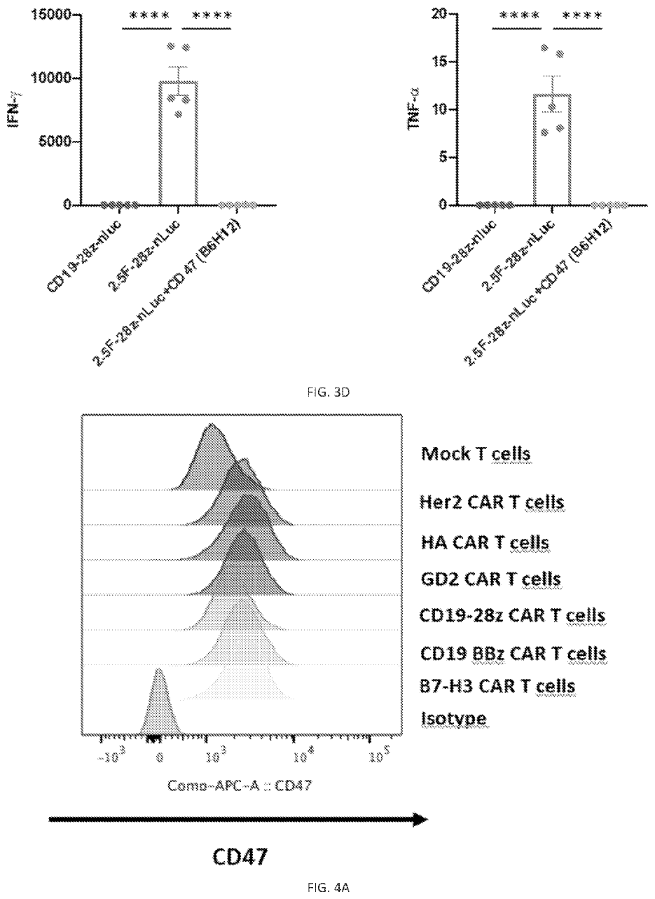

To better understand CAR T cell location and kinetics, nanoluc protein was fused to the CAR construct which allowed CAR T cell imaging. T cell imaging revealed that T cell depletion occurs very rapidly, i.e., within 2 days after T cell administration the CAR signal had mostly disappeared (FIG. 2). Macrophage depletion led to increased bioluminescence signal from the CAR T cells, suggesting that in the absence of macrophages CD47 blockade is ineffective (FIG. 2). CD47 blockade also diminished CAR-mediated toxicity, as shown in FIG. 3.

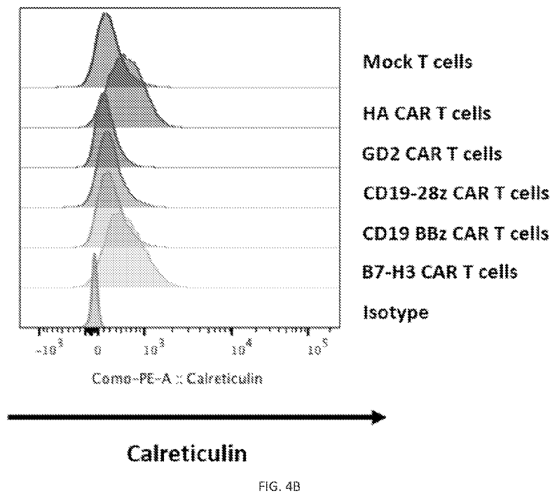

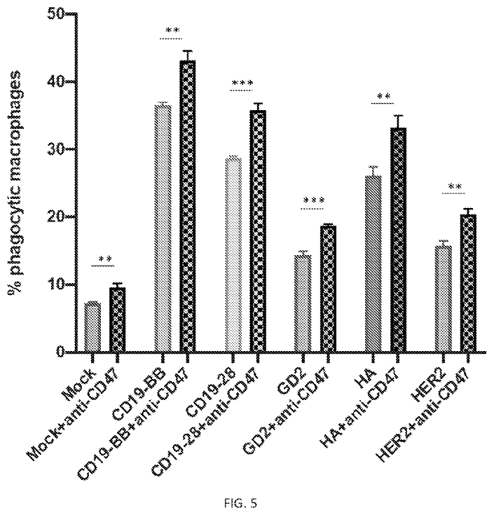

Phagocytosis is a balancing act determined by pro- and anti-phagocytic signals. CAR T cells express the pro-phagocytic signal calreticulin and anti-phagocytic signal CD47 (FIG. 4). It was hypothesized that CD47 blockade would lead to phagocytosis of CAR T cells. Indeed, in an in vitro phagocytosis assay, CD47 blockade significantly increased phagocytosis of CAR T cells by macrophages compared to control (FIG. 5).

These results suggest that anti-CD47 may be utilized as a safety switch to eliminate CAR T cells when needed.

### Example 2

This example demonstrates that CAR T cells acquire a “don't-eat-me” phenotype upon activation.

The kinetics of expression of genes involved in modulating phagocytic activity and interaction between macrophage/T cells was investigated. Human T cells from three healthy donors were transduced with a retroviral construct expressing the FCM63 CAR which targets CD19 (van der Stegen et al., Nat Rev Drug Discov., 14(7): 499-509(2015); doi: 10.1038/nrd4597. PMID: 26129802).

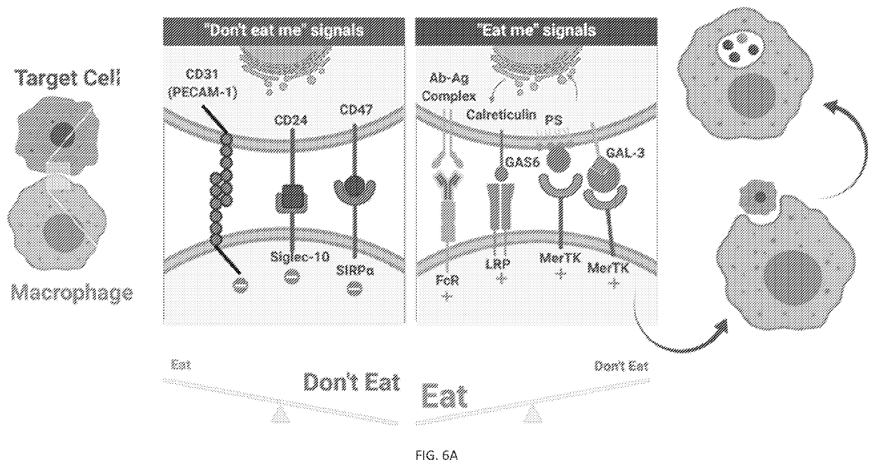

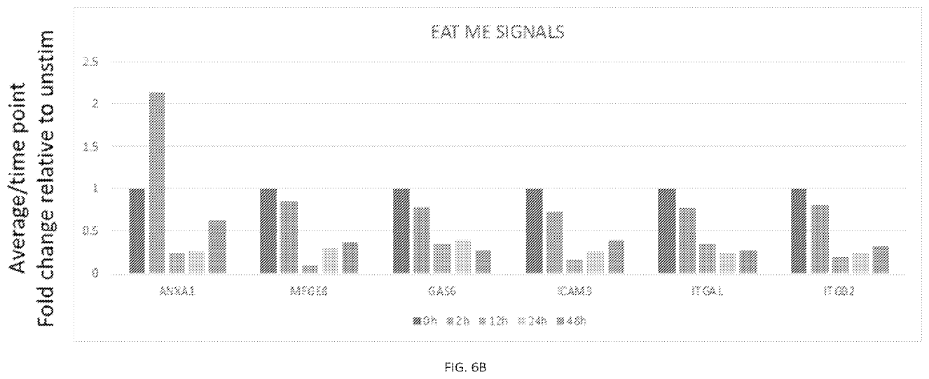

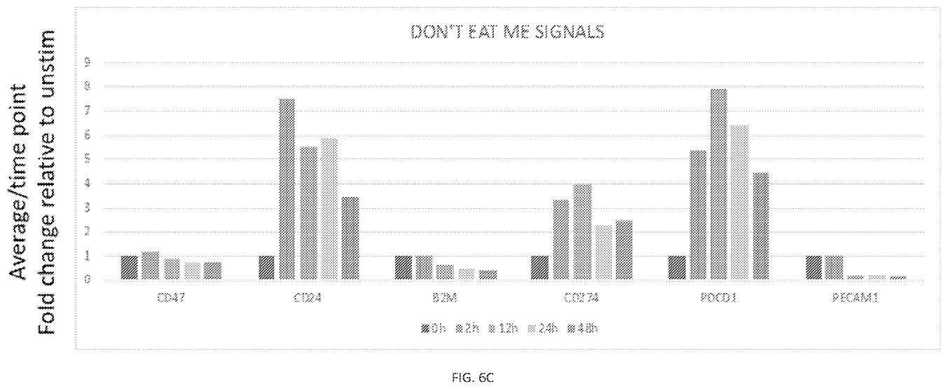

After 10 days in culture, CAR T cells were stimulated with an FCM63 anti-idiotype antibody and samples were collected at 2 hours (h), 12 h, 24 h, 48 h and 72 h later. Bulk RNA was extracted and sequenced. Upon stimulation, genes encoding “eat me signals” showed quick downregulation that was sustained throughout the entire time course. Opposite trends were observed for genes encoding “don't eat me” signals, with robust upregulations of expression that were also sustained for the duration of the experiment (72 h) (FIG. 6). Also observed was a very rapid upregulation of the CD40 ligand, CD40LG. Binding of CD40LG on the T cell to CD40 on the macrophage is necessary for the activation of cytokine production by T cells upon TCR activation by antigen presented in the context of MHC.

These data show that activated T cells acquire a “don't eat me” phenotype that allows quick unchecked expansion, and that they may be susceptible to drugs targeting such molecules.

### Example 3

This example demonstrates that overexpression of CD47 increases CAR T cell activity.

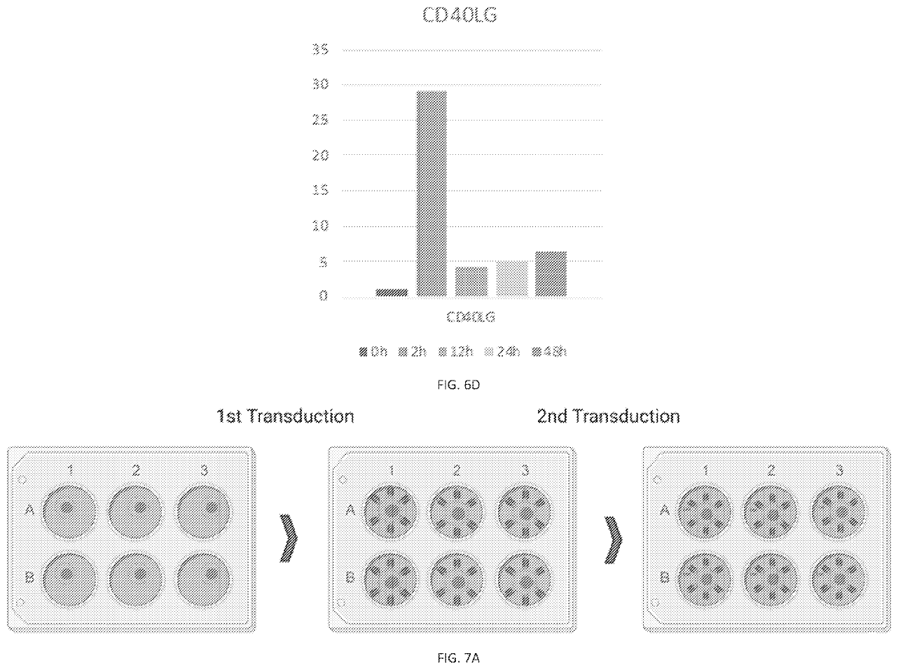

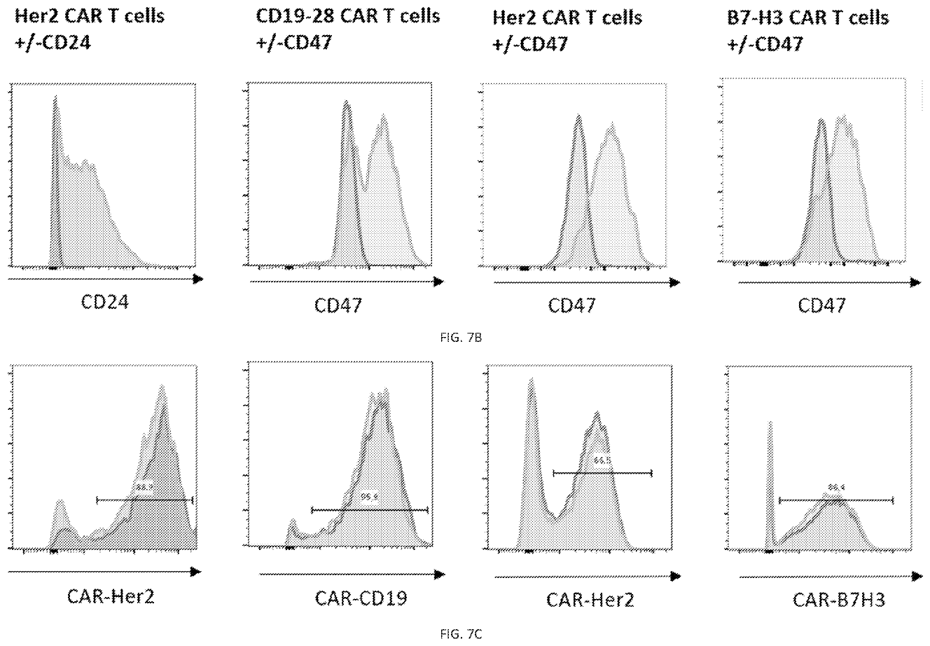

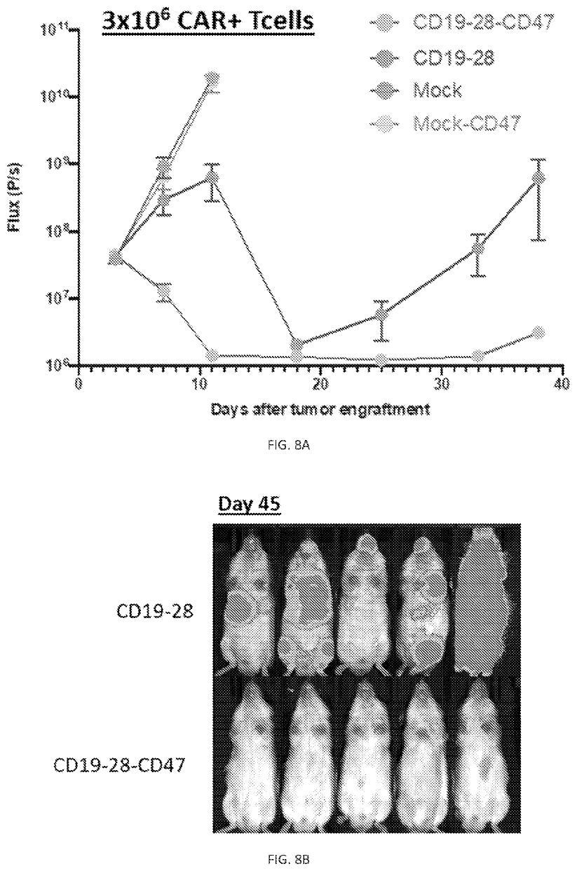

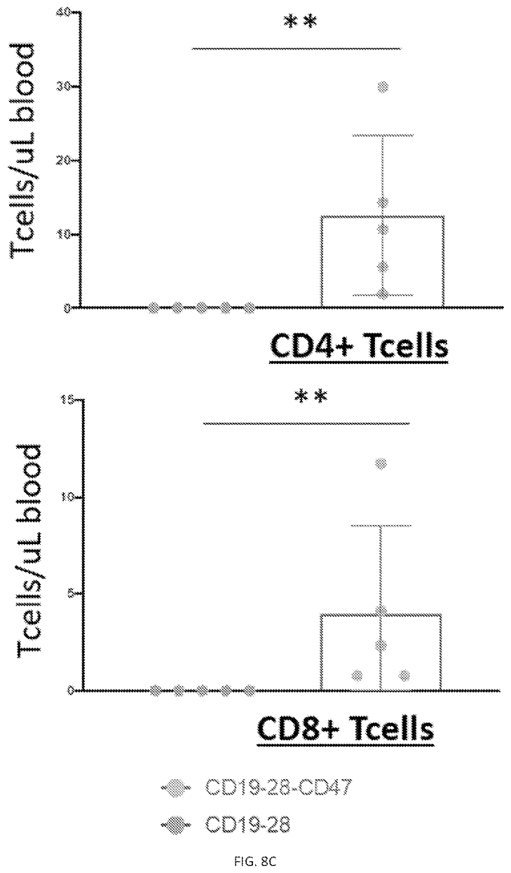

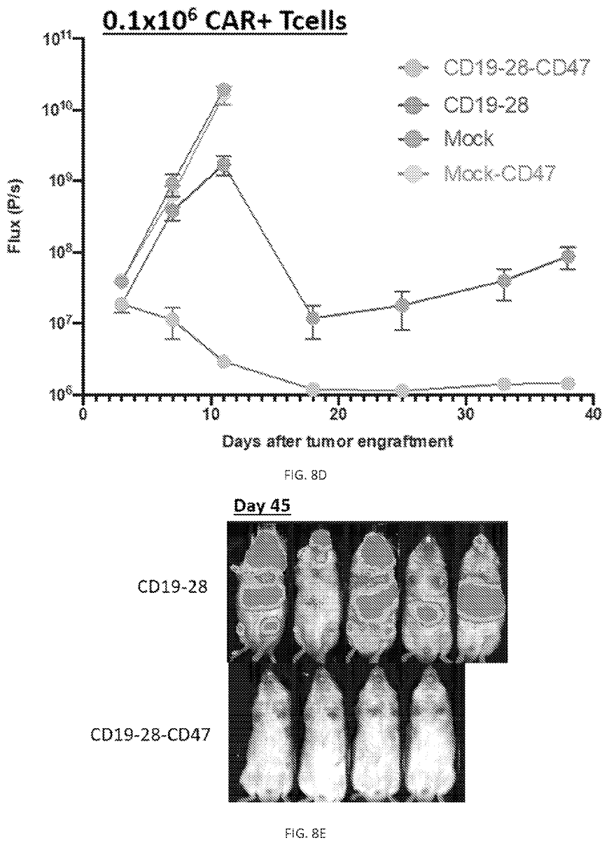

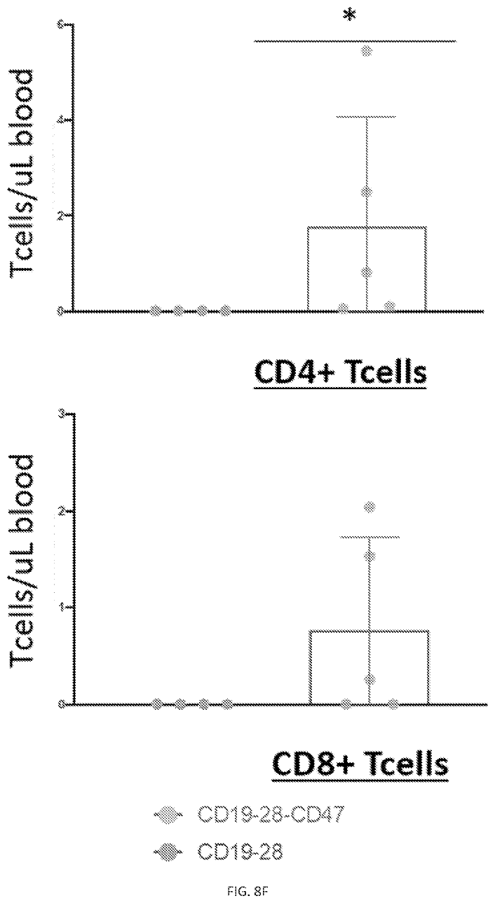

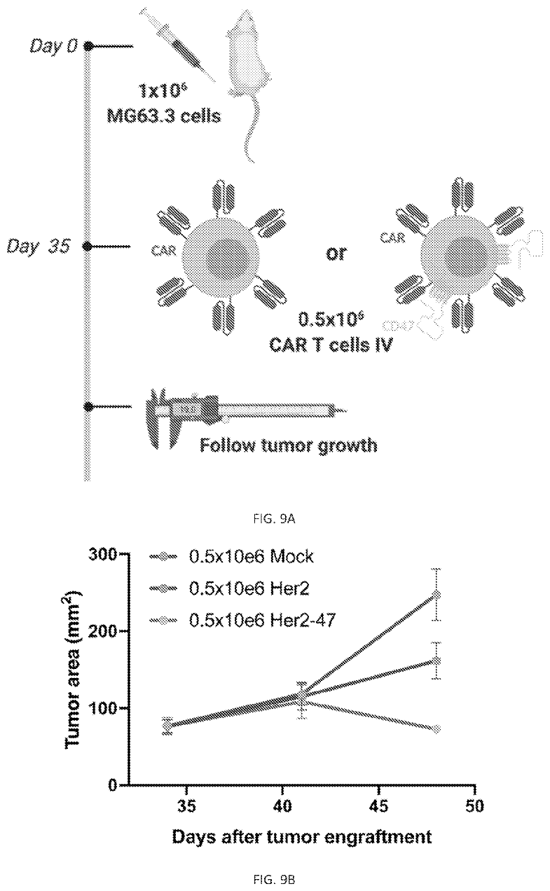

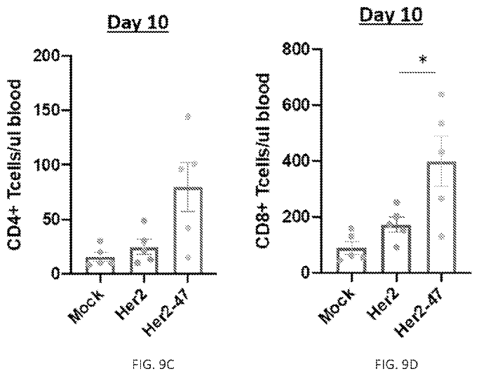

Since blocking CD47 with a monoclonal antibody depletes CAR T cells, it was investigated whether CD47 overexpression can protect CAR T cells from macrophage depletion. Different CARs were screened for CD47 expression during manufacturing and all of them were found to express CD47. Next, CD47 was retrovirally overexpressed and expression was assessed by flow cytometry. CD47 overexpression was efficiently achieved in several CAR T cells without deleterious effect on the level of CAR expression (FIG. 7). Anti-tumor activity of CD47-overexpressing CAR T cells targeting CD19 and HER2 was assessed in a mouse model of leukemia (FIG. 8) and osteosarcoma (FIG. 9), respectively. CD47-overexpressing CD19-CAR T cells demonstrated superior activity compared to the constitutive CAR. At a dose of 3×10⁶, CD47-regular CD19-CAR T cells (CD47reg) first regressed tumors before they all relapsed, whereas CD47-overexpressing CAR T cells (CD47overexp) induced a permanent cure. At a low dose of 0.1×10⁶ CAR+ T cells CD47 constitutive CD19-CAR T cells demonstrated slight slow-down of tumor growth, whereas CD47overexp CD19-CAR T cells cured treated mice. At both doses, CD47 overexpression led to superior CAR T cell function compared to the constitutive CAR (FIG. 8). Furthermore, 43 days after one time CAR T cell treatment persisting T cells were detected only in the mice which were treated with the CD47overexp CAR T cells, whereas there were no persisting CAR T cells detected in the mice which were treated with regular CAR T cells (FIG. 8). Reduced phagocytosis of CD47 overexpressing CAR T cells by phagocytes presumably leads to increased persistence and increases CAR T cell efficacy.

In the osteosarcoma model, 0.5×10⁶ CD47-overexpressing HER2 CAR T cells led to rapid cure and demonstrated superior response compared to the constitutive CAR (FIG. 9). Ten days after CAR T cell administration more CAR T cells were found in the blood of the CD47 overexpressing HER2 CAR T cells, indicating better expansion and survival (FIG. 9).

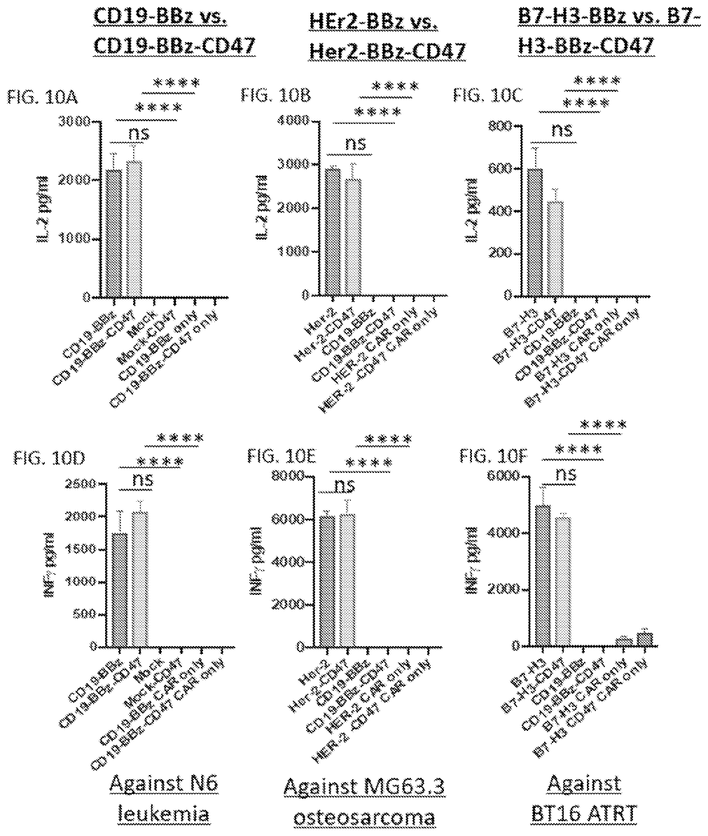

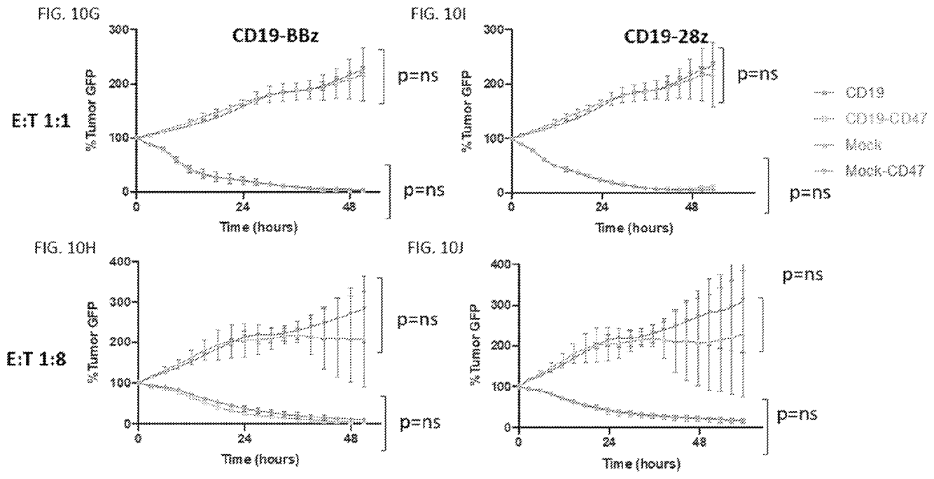

Further, in the absence of macrophages, killing capacity and cytokine release of INFgamma and IL-2 was similar between CD47 overexpressing and regular CAR T cells in vitro, demonstrating that T cell function itself is not altered upon CD47 overexpression (FIG. 10).

These results show that overexpressing CD47 on CAR T cells protects them from macrophage mediated clearance and leads to enhanced expansion and persistence and superior efficacy.

### REFERENCES

All references, including publications, patent applications, and patents, cited herein are hereby incorporated by reference to the same extent as if each reference were individually and specifically indicated to be incorporated by reference and were set forth in its entirety herein.
1. Majzner, R. G. & Mackall, C. L. Clinical lessons learned from the first leg of the CAR T cell journey. Nat Med 25, 1341-1355, doi:10.1038/s41591-019-0564-6 (2019).
2. Qian, B. Z. & Pollard, J. W. Macrophage diversity enhances tumor progression and metastasis. Cell 141, 39-51, doi:10.1016/j.cell.2010.03.014 (2010).
3. Jaiswal, S. et al. CD47 is upregulated on circulating hematopoietic stem cells and leukemia cells to avoid phagocytosis. Cell 138, 271-285, doi:10.1016/j.cell.2009.05.046 (2009).
4. Li, W. Eat-me signals: keys to molecular phagocyte biology and “appetite” control. J Cell Physiol 227, 1291-1297, doi:10.1002/jcp.22815 (2012).
5. Barkal, A. A. et al. CD24 signalling through macrophage Siglec-10 is a target for cancer immunotherapy. Nature 572, 392-396, doi:10.1038/s41586-019-1456-0 (2019).
6. Hutter, G. et al. Microglia are effector cells of CD47-SIRPalpha antiphagocytic axis disruption against glioblastoma. Proc Natl Acad Sci USA 116, 997-1006, doi:10.1073/pnas.1721434116 (2019).
7. Mohanty, S., Aghighi, M., Yemeni, K., Theruvath, J. L. & Daldrup-Link, H. E. Improving the efficacy of osteosarcoma therapy: combining drugs that turn cancer cell ‘don't eat me’ signals off and ‘eat me’ signals on. Mol Oncol 13, 2049-2061, doi:10.1002/1878-0261.12556 (2019). 8. Advani, R. et al. CD47 Blockade by Hu5F9-G4 and Rituximab in Non-Hodgkin's Lymphoma. N Engl J Med 379, 1711-1721, doi:10.1056/NEJMoa1807315 (2018).
9. Sikic, B. I. et al. First-in-Human, First-in-Class Phase I Trial of the Anti-CD47 Antibody Hu5F9-G4 in Patients With Advanced Cancers. J Clin Oncol 37, 946-953, doi:10.1200/JCO.18.02018 (2019).
10. Xia, A., Zhang, Y., Xu, J., Yin, T. & Lu, X. J. T Cell Dysfunction in Cancer Immunity and Immunotherapy. Front Immunol 10, 1719, doi:10.3389/fimmu.2019.01719 (2019).

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An engineered lymphocyte which overexpresses one or more anti-phagocytic signaling proteins.

2. The engineered lymphocyte of claim 1, wherein the one or more anti-phagocytic signaling proteins is selected from CD47, CD24 and CD31.

3. The engineered lymphocyte of claim 2, which overexpresses CD47.

4. The engineered lymphocyte of any one of claims 1-3, which is a T cell.

5. The engineered lymphocyte of any one of claims 1-4, which expresses a chimeric antigen receptor (CAR) polypeptide.

6. The engineered lymphocyte of claim 5, wherein the CAR polypeptide comprises an antigen binding domain, a transmembrane domain, at least one co-stimulatory signaling domain, an intracellular signaling domain.

7. The engineered lymphocyte of claim 6, wherein the antigen binding domain specifically binds to a CD19 antigen or a human epidermal growth factor receptor 2 (HER2).

8. The engineered lymphocyte of any one of claims 1-3, which is a natural killer (NK) cell.

9. A composition comprising the engineered lymphocyte of any one of claims 1-8 and a pharmaceutically acceptable carrier.

10. A method of inducing an immune response against one or more cancer cells, which comprises contacting one or more cancer cells with the composition of claim 9, whereupon an immune response against the one or more cancer cells is induced.

11. The method of claim 10, wherein the one more cancer cells are in vitro.

12. The method of claim 11, wherein the one or more cancer cells are in vivo.

13. The method of claim 12, wherein the one or more cancer cells are in a human.

14. Use of an engineered lymphocyte of any one of claims 1-8 or a composition of claim 9 for the treatment of cancer.

15. A method of inhibiting immune clearance of genetically engineered T cells in a subject, which method comprises:
administering genetically engineered T cells that overexpress one or more anti-phagocytic signaling proteins to a subject in need thereof, whereby the one or more anti-phagocytic signaling proteins are overexpressed by the genetically engineered T cells and immune clearance of the genetically engineered T cells is inhibited.

16. The method of claim 15, which inhibits macrophage-mediated immune clearance of the genetically engineered T cells.

17. The method of claim 15 or claim 16, wherein the one or more anti-phagocytic signaling proteins is selected from CD47, CD24 and CD31.

18. The method of claim 17, where the anti-phagocytic signaling protein is CD47.

19. The method of any one of claims 15-18, wherein the genetically engineered T cells further express a chimeric antigen receptor (CAR) polypeptide.

20. The method of claim 19, wherein the CAR polypeptide specifically binds to a CD19 antigen or a human epidermal growth factor receptor 2 (HER2) expressed on the surface of cancer cells.

21. A method of depleting engineered T cells in a subject in need of T cell depletion, which method comprises administering to a subject who has received engineered T cells an agent that inhibits the activity of one or more anti-phagocytic signaling proteins expressed by the engineered T cells.

22. The method of claim 21, wherein the one or more anti-phagocytic signaling proteins is selected from CD47, CD24 and CD31.

23. The method of claim 22, wherein the anti-phagocytic signaling protein is CD47.

24. The method of any one of claims 21-23, wherein the agent is an antibody.

25. The method of claim 24, wherein the agent is an anti-CD47 monoclonal antibody.

26. The method of any one of claims 21-25, wherein the engineered T cells express a chimeric antigen receptor (CAR) polypeptide.

27. The method of any one of claims 21-26, wherein the engineered T cells induce toxicity in the subject.

28. The method of claim 27, wherein the toxicity is cytokine release syndrome (CRS) and/or neurologic toxicity.

29. Use of an agent that inhibits the activity of one or more anti-phagocytic signaling proteins for depleting engineered T cells in a subject.
